(19) United States
(12) Patent Application Publication
(10) Pub. No.: US 2025/0072934 A1
(43) Pub. Date: Mar. 6, 2025

(54) SOFT TISSUE ACCESS SUPPORT DEVICES AND METHODS

(71) Applicant: EDWARDS LIFESCIENCES CORPORATION, Irvine, CA (US)

(72) Inventor: Yuval Kasher, Kfar Shmuel (IL)

(21) Appl. No.: 18/948,838

(22) Filed: Nov. 15, 2024

Related U.S. Application Data
(63) Continuation of application No. PCT/US2023/023874, filed on May 30, 2023.
(60) Provisional application No. 63/347,466, filed on May 31, 2022.

(51) Int. Cl. A61B 17/34; A61B 17/00

(52) U.S. Cl. CPC A61B 17/3423; A61B 2017/00867; A61B 2017/3419; A61B 2017/3425; A61B 2017/3486

(57) ABSTRACT

Disclosed are various devices and methods for providing tissue access support. One exemplary device embodiment includes an elongated body having distal and proximal ends and extending along a longitudinal axis and defining a first lumen therein. A sealing member is coupled adjacent to the distal end of the elongated body and structured to seal to a first surface of a pericardial tissue. A support tube is provided having distal and proximal ends and extending along a longitudinal axis and defining a second lumen therein, wherein the support tube is slidably arranged in the lumen of the elongated body between first and second sliding positions. A securement member is coupled to the support tube and structured to engage at least a second opposing surface of the pericardial tissue. A guidewire is positioned in the second lumen of the support tube and includes a puncturing tip shaped to penetrate pericardial tissue.

### RELATED APPLICATIONS

This application is a continuation application of International Patent Application No. PCT/US2023/023874, filed May 30, 2023, which claims the benefit of U.S. Provisional Application No. 63/347,466, filed on May 31, 2022. Each of the foregoing patent applications are hereby incorporated by reference in their entireties.

### TECHNICAL FIELD

This disclosure is directed to devices and methods for accessing soft tissue, such as for example, an appendage, a vessel, a membrane inner wall, a pericardium, and providing supportive structure for subsequent deployment of device with a reduction or elimination of additional traumatic injury to the soft tissue and the minimization or elimination of bleeding or drainage of fluids.

### BACKGROUND

Accessing various compartments of the mammalian body, for example, the pericardial cavity to relieve pericardial restraint, transmural ablation procedures, valve replacement and repair, etc. typically requires puncturing and inserting devices in soft tissue, such as the right atrium (RA), the right atrial appendage (RAA), the coronary sinus (CS), the pericardial membrane, for example. Subsequent manipulation of such devices after puncturing such tissue can result in trauma to the punctured tissue requiring hemostasis to occur.

### SUMMARY

In one example, a soft tissue access support device is provided, comprising: an elongated body having a distal end and a proximal end along a longitudinal axis defining a lumen, the distal end coupled to a sealing member; a support tube having a distal end and a proximal end along a longitudinal axis, the distal end coupled to a securement member; the support tube is slidably arranged in the elongated body, wherein the elongated body is structured so as to facilitate placement of the sealing member in sealable engagement with a portion of a first surface of a soft tissue; and wherein said support tube is structured, such that when slid from a first sliding position to a second sliding position, the support tube enters an access opening in the soft tissue and the securement member attached to the distal end of the support tube is in engagement with a second opposing surface of the soft tissue.

In one aspect, the elongated body is a multi-lumen catheter. In another aspect, alone or in combination with any of the previous aspects, the elongated body is steerable. In another aspect, alone or in combination with any of the previous aspects, the support tube is a multi-lumen catheter. In another aspect, alone or in combination with any of the previous aspects, the support tube is steerable.

In another aspect, alone or in combination with any of the previous aspects, the securement member comprises one or more anchors. In another aspect, alone or in combination with any of the previous aspects, the one or more anchors comprises an inflatable device. In another aspect, alone or in combination with any of the previous aspects, the securement member is two or more reversibly collapsible, radially expanding shape-memory legs.

In another aspect, alone or in combination with any of the previous aspects, the sealing member is an thermoplastic or thermoset elastomeric, low compression set material. In another aspect, alone or in combination with any of the previous aspects, the sealing member is selected from thermoplastic elastomers, EPDM rubbers, thermoset rubbers, polysilicones, polysiloxanes, polyurethanes, polyvinyl chlorides, styrene-ethylene-butylene-styrenes, polytetrafluoroethylenes, and derivatives, copolymers, and blends thereof. In another aspect, alone or in combination with any of the previous aspects, the sealing member is an O-ring. In one embodiment, the sealing member is an inflatable O-ring.

In another aspect, the soft tissue is selected from an appendage, a vessel, a membrane inner wall, or a pericardium, the pericardium having a parietal layer surrounding a pericardial cavity. In one aspect, the elongated body is structured so as to facilitate placement of the sealing member in sealable engagement with a portion of a first surface of the pericardium, and wherein the support tube is structured, such that when slid from a first sliding position to a second sliding position, the support tube enters the access opening in the pericardium and the securement member attached to the distal end of the support tube is in engagement with a portion of the second opposing surface of the parietal layer such that the sealing member and the securement member create a seal relative to the access opening in the pericardium.

In another aspect, alone or in combination with any of the previous aspects, the device further comprises a transection device is coupled to a distal end of a catheter. In another aspect, alone or in combination with any of the previous aspects, the transection device comprises a blade or electrode or combination, capable of cutting soft tissue.

In another aspect, alone or in combination with any of the previous aspects, the elongated body, support tube, sealing member, securement member, or transection device comprises radiopaque material randomly distributed or arranged in a pattern. In another aspect, alone or in combination with any of the previous aspects, the device is sterilized.

In a second example, a soft tissue access support device is provided, the device comprising: an elongated body having a distal end and a proximal end along a longitudinal axis defining a lumen; and a support tube having a distal end and a proximal end along a longitudinal axis, the distal end coupled to a securement member; the support tube is slidably arranged in the elongated body, wherein in a first configuration, the elongated body and the support tube are both engaged with a portion of an first surface of the soft tissue; and wherein in a second configuration, the securement member is securely engaged with at least a portion of a second opposing surface of the soft tissue.

In one aspect, the elongated body is a multi-lumen catheter. In another aspect, alone or in combination with any of the previous aspects, the elongated body is steerable. In another aspect, alone or in combination with any of the previous aspects, the support tube is a multi-lumen catheter. In another aspect, alone or in combination with any of the previous aspects, the support tube is steerable.

In another aspect, alone or in combination with any of the previous aspects, the securement member comprises an anchor. In another aspect, alone or in combination with any of the previous aspects, the anchor comprises a helical coil.

In another aspect, alone or in combination with any of the previous aspects, the securement member is engaged with both the first surface and at least a portion of the second opposing surface of the soft tissue.

In another aspect, alone or in combination with any of the previous aspects, the access opening in the soft tissue is defined by a tunnel extending from the first surface to the second opposing surface of the soft tissue, wherein the securement member is engaged with the second opposing surface of the soft tissue and at least a portion of the tunnel defining the access opening.

In another aspect, alone or in combination with any of the previous aspects, the support member is slidably received through the helical coil.

In another aspect, alone or in combination with any of the previous aspects, the elongated body comprises a plurality of tapered segments that coalesce. In another aspect, alone or in combination with any of the previous aspects, the plurality of tapered segments are separated by the support tube in the second configuration.

In another aspect, alone or in combination with any of the previous aspects, the soft tissue is a pericardium having a parietal layer surrounding a pericardial cavity, wherein the elongated body is structured so as to engage a portion of a first surface of the pericardium, and wherein said support tube is structured, and wherein in the second configuration, the securement member is securely engaged with at least a portion of a second opposing surface of the parietal layer.

In another aspect, alone or in combination with any of the previous aspects, the device further comprises a pericardial transection device coupled to a distal end of a catheter. In another aspect, alone or in combination with any of the previous aspects, the pericardial transection device comprises a blade or electrode or combination, capable of cutting pericardial tissue.

In another aspect, alone or in combination with any of the previous aspects, the elongated body, support tube, securement member, or transection device comprises radiopaque material randomly distributed or arranged in a pattern. In another aspect, alone or in combination with any of the previous aspects, the device is sterilized.

In a third example, a method is provided, the method comprising: (i) presenting a distal end of a soft tissue access support device according to any one of the previous claims; (ii) slidably extending the support tube from the soft tissue access support device and through an opening in the first surface of the soft tissue; (iii) presenting the securement member to at least a portion of a an opposing surface of the soft tissue; and (iv) coupling the soft tissue access support device to the soft tissue.

In one aspect, the method further comprises introducing a device to the soft tissue through the support tube. In another aspect, alone or in combination with any of the previous aspects, further comprising introducing a device to the soft tissue through the securement member.

In another aspect, alone or in combination with any of the previous aspects, the activating the sealing member comprises retracting support tube and compressing a portion of the soft tissue between the sealing member and a securement member.

In another aspect, alone or in combination with any of the previous aspects, the securement member comprises one or more anchors. In another aspect, alone or in combination with any of the previous aspects, the one or more anchors comprises an inflatable device. In another aspect, alone or in combination with any of the previous aspects, the anchor comprises a helical coil. In another aspect, alone or in combination with any of the previous aspects, the securement member is two or more reversibly radially expanding shape-memory legs.

In another aspect, alone or in combination with any of the previous aspects, the elongated body is a steerable catheter. In another aspect, alone or in combination with any of the previous aspects, the support tube is a steerable catheter.

In another aspect, alone or in combination with any of the previous aspects, the method further comprises a pericardial access device coupled to a distal end of a catheter. In another aspect, alone or in combination with any of the previous aspects, the pericardial transection device comprises a blade or electrode.

In another aspect, alone or in combination with any of the previous aspects, the sealing member is an elastomeric, low compression set polymer. In another aspect, alone or in combination with any of the previous aspects, the sealing member is selected from thermoplastic elastomers, EPDM rubbers, thermoset rubbers, polysilicones, polysiloxanes, polyurethanes, polyvinyl chlorides, styrene-ethylene-butylene-styrenes, polytetrafluoroethylenes, and derivatives, copolymers, and blends thereof.

In another aspect, alone or in combination with any of the previous aspects, the soft tissue access support device is presented subxiphoidally to the first surface. In another aspect, alone or in combination with any of the previous aspects, the soft tissue access support device is introduced transvascularly to the first surface. In another aspect, alone or in combination with any of the previous aspects, the soft tissue access support device is presented transvascularly to the first surface via a Superior Vena Cava. In another aspect, alone or in combination with any of the previous aspects, the soft tissue access support device is presented transvascularly to the first surface via an Inferior Vena Cava. In another aspect, alone or in combination with any of the previous aspects, the soft tissue access support device is presented transvascularly to the first surface via a coronary sinus. In another aspect, alone or in combination with any of the previous aspects, the method further comprises providing access to at least one cavity through the soft tissue.

In a fourth example, a method of manipulating a transection device is provided, the method comprising: (i) providing a soft tissue access support device according to any one of the previous aspects; (ii) slidably extending the support tube from the soft tissue access support device; and (iii) manipulating the securement member with a controller.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1B of FIG. 1A depicting the serosal, visceral, fibrous layers and adipose tissue of the parietal pericardium, including the pericardial cavity.

FIG. 2 is a depiction of an exemplary soft tissue access support device, as disclosed and described herein.

FIGS. 3A and 3B are a depictions of the exemplary soft tissue access support device of FIG. 2 device providing access to a pericardium, as disclosed and described herein.

FIGS. 4A and 4B are additional depictions of the exemplary soft tissue access support device of FIG. 2 device providing access to a pericardium, as disclosed and described herein.

FIG. 5 is a depiction of another exemplary soft tissue access support device providing access to soft tissue, as disclosed and described herein.

FIG. 6 is a simplified diagram of a transcatheter approach to soft tissue, e.g., the pericardial cavity, as disclosed and described herein.

FIG. 7 is a simplified diagram of an alternative transcatheter approach to the soft tissue, as disclosed and described herein.

FIG. 8 is a simplified diagram of an exemplary incision path in soft tissue by using a soft tissue access support device in accordance with methods disclosed and described herein.

### DETAILED DESCRIPTION

The present disclosure provides for providing access through soft tissue, for example, to allow access of a medical device with mechanical support as well as maintaining and/or contributing to hemostasis or supplying sufficient hemostasis during manipulation of such devices during medical procedures. The present disclosure also provides for providing access through soft tissue via a transcatheter device approach without the need for leaving an occluder or other hemostasis implant behind. The presently disclosed access device provides reinforcement for a variety of medical devices that puncture or have punctured through soft tissue, such as membranes, for example pericardium membrane, muscle tissue, and the like. The presently disclosed access support devices provide an access through a reinforcement structure that is secured to the tissue about a puncture site and thus allow the introduction of devices and subsequent manipulation of such devices without causing additional trauma and/or stress and strain to the puncture site.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “cutting surface” is inclusive of one or more of an edge of a sharpened blade or the surface of an electrode structured to receive sufficient current or radio frequency energy (RF) to ablate, burn, vaporize or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a transcatheter device or catheter, and the application of a directional force sufficient to cut or separate the tissue, the force being substantially in a direction towards the proximal end of the transcatheter device or catheter, for example, by pulling the transcatheter device or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an end point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “preserved ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “heart dysfunction” is inclusive of the ordinary and customary meaning to one of ordinary skill in medical and surgical arts, for example, heart failure or congestive heart failure.

As used herein the phrase “pericardial transection device” is inclusive of a device with a cutting surface, for example an edge of a blade or a surface of an electrode.

As used herein the phrase “pericardial incision assembly” and “incision assembly” are used interchangeable and refer to an assemblage that includes a pericardial transection device.

As used herein the phrase “transcatheter device” is inclusive of a catheter structured with at least one lumen comprising a medical instrument, device, or component thereof, for example, a pericardial transection device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein, “delta pulmonary capillary wedge pressure, Δ(PCWP)” is the difference between pulmonary capillary wedge pressure (PCWP) measured on volume challenge (i.e. leg raise and/or fluid administration) and PCWP at rest.

As used herein the phrase “puncturing tip” is inclusive of an atraumatic object suitable for puncturing or penetrating tissue without substantial trauma to or bleeding from the vicinity of the picture or penetration.

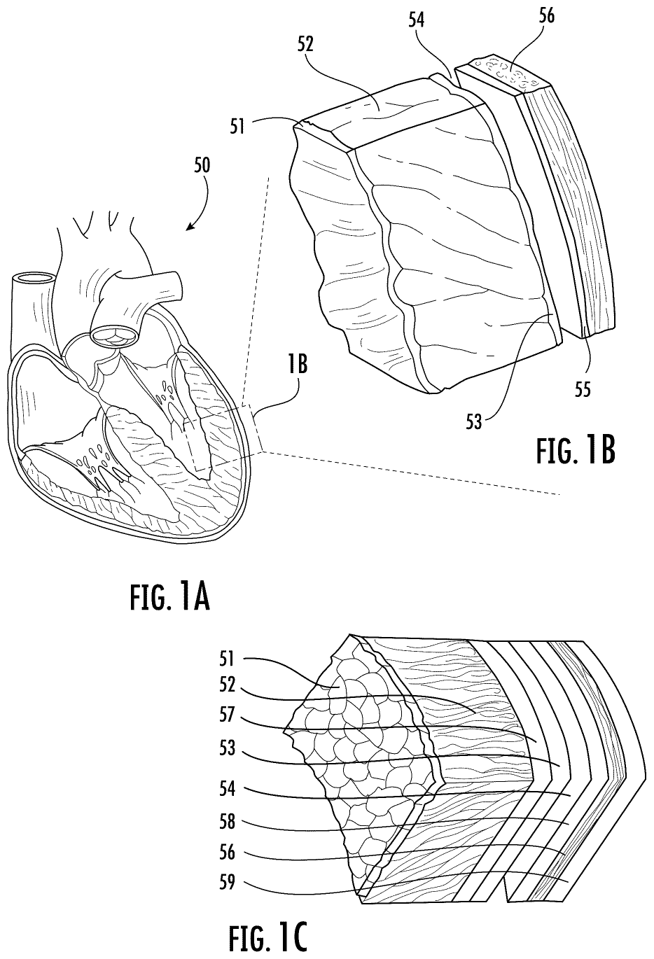

With reference to FIGS. 1A, 1B, 1C, and section 1B, layers of a heart wall of a heart 50, from inside-out, being the endocardium 51, the myocardium 52, epicardial adipose tissue 57, the visceral layer 53 of the serous pericardium, the pericardial cavity 54, the parietal layer 55 of the serous pericardium 58, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium.

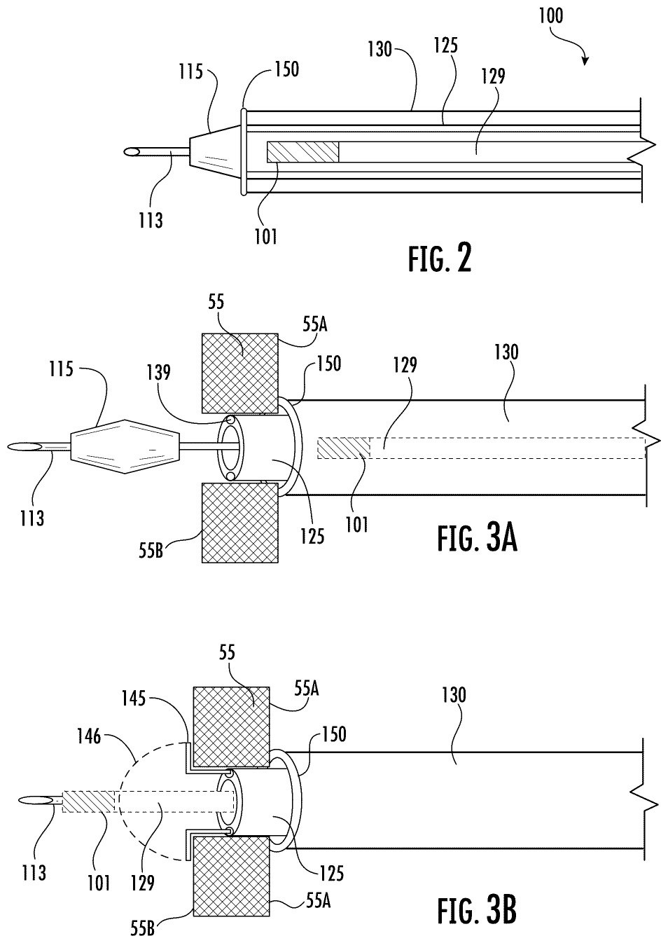

With reference to FIGS. 2, 3A, 3B soft tissue access support device 100 is shown having an elongated body 130 having a distal end and a proximal end along a longitudinal axis defining a lumen, the distal end coupled to a sealing member 150. Distal end of the elongated body 130 is shown together with an introducer/dilator 115 which is structured to receive a guidewire 113, which may be a puncturing device and provide initial access to the soft tissue that the access support device 100 will employ.

Support tube 125 having a distal end and a proximal end along a longitudinal axis, has distal end coupled to a securement member 146. Support tube 125 slidably arranged in the elongated body 130, where in a first sliding position, the sealing member 150 and a portion of a first surface 55A (first surface and anterior surface used interchangeably) of a pericardium are sealably engaged. In a second sliding position, the securement member 146 and a portion of an opposing surface 55B of the pericardium are securably engaged. In one example, the support tube 125 is slid forward through the first surface 55A and opposing surface 55B, with the securement member 146 being deployed and/or activated. Subsequently, support tube 125 is slid rearwardly so that securement member 146 and sealing member 150 grasp and/or pinch pericardium and provide for a secure configuration for extending support tube 125.

The elongated body 130 can be a multi-lumen catheter. The elongated body 130 can be steerable. In one example, the support tube 125 is a multi-lumen catheter, having lumen 139 for slidably receiving/extending securement member 146. Support tube 125 can be steerable. As shown in FIG. 3B, securement member 146 comprises one or more anchors 145, e.g., shape-memory reversibly self-radially expanding legs and the like. In another example, the one or more anchors 146 comprises an inflatable device, such as a balloon, which can be O-ring shaped.

Sealing member 150 can be any medical grade thermoplastic or thermoset elastomeric, low compression set material. In one example, sealing member 150 is selected from thermoplastic elastomers, EPDM rubbers, thermoset rubbers, polysilicones, polysiloxanes, polyurethanes, polyvinyl chlorides, styrene-ethylene-butylene-styrenes, polytetrafluoroethylenes, and derivatives, copolymers, and blends thereof. In another example, the sealing member 150 is an O-ring.

As shown in FIG. 2, pericardial transection device 101 is coupled to a distal end of a catheter 129. Pericardial transection device 101 can comprise a blade, or electrode, or combination, capable of cutting pericardial tissue. A variety of pericardial transection devices can be used provided they are of an outer diameter less than the inner diameter of the support tube 125. As shown in FIG. 3B, catheter one 29 and pericardial transection device 101 can be deployed over the wire (OTW) using guidewire 113 received by a lumen of a multi-lumen catheter.

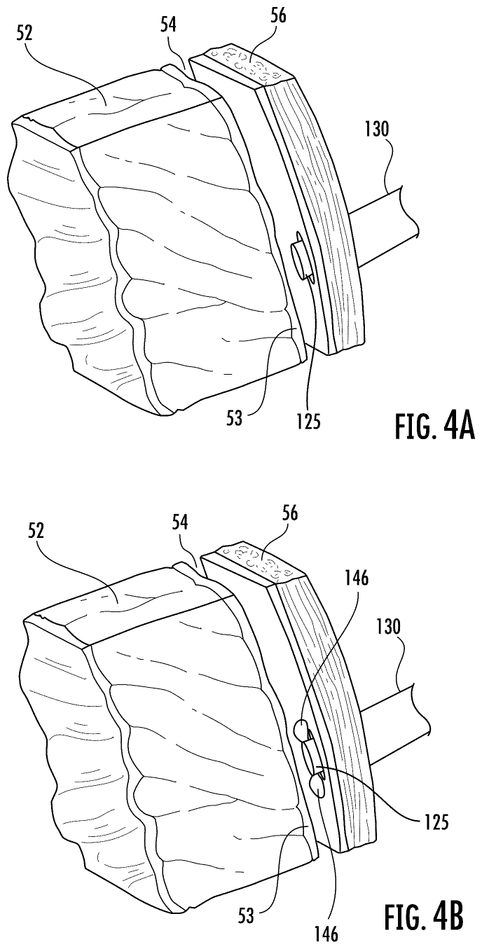

With reference to FIGS. 4A, 4B, perspective views of soft tissue access support device 100 is shown penetrating through first and opposing surfaces 55A, 55B, respectively, of pericardium with deployment of support tube and securement member 146 about the opposing surface 55B for providing access to the pericardial cavity 54 of a flexible catheter 129 having coupled to its distal end a pericardial transection device 101.

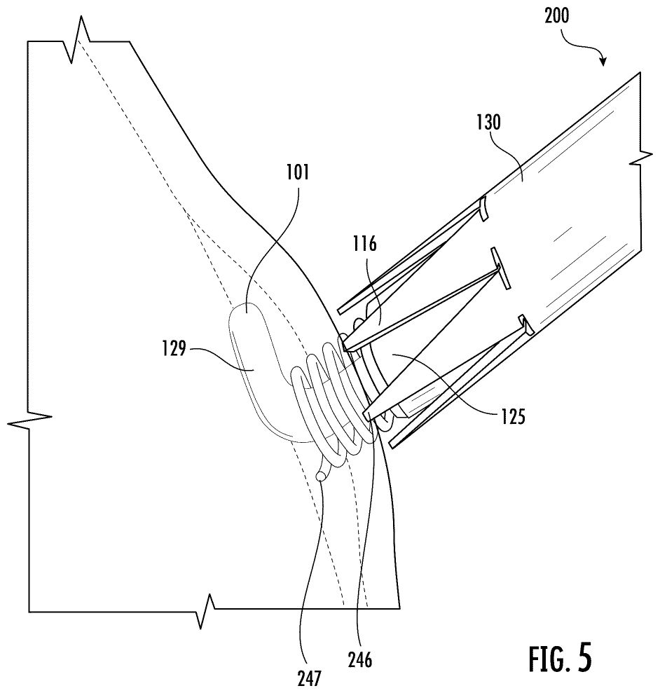

With reference to FIG. 5, alternative access support device 200 is shown having elongated body 130 with a distal end and a proximal end along a longitudinal axis defining a lumen. The distal end of the elongated body 130 is structured to be followed by an introducer/dilator 115 set that prepares the entry site to receive the body 130. Support tube 125 having a distal end and a proximal end along a longitudinal axis, the distal end coupled to an securement member 246, is slidably arranged in the lumen of elongated body 130, where, in a first configuration, the introducer and support tube 125 are both engaged with a portion of an first surface 55A of a pericardium, and where, in a second configuration, the securement member 246 is securely engaged with the first surface 55A and a portion of an opposing surface 55B of the pericardium.

In one example, elongated body 130 of access device 200 is a multi-lumen catheter. The elongated body 130 can be steerable. The elongated body 130 can be a multi-lumen catheter.

In one example, the support tube 125 of access support device 200 is a multi-lumen catheter. Support tube 125 can be steerable.

In one example, securement member 146 comprises anchor 246 to secure the elongated body 130 about the tissue. As shown in FIG. 5, the anchor 246 comprises a helical coil terminating in a tip 247 structured to engage the tissue, and upon rotation, urge anchor 246 into the tissue. As shown, the securement member 246 is helically engaged with the first surface 55A and a portion of an opposing surface 55B of the pericardium. Rotation of the anchor 246 can be achieved using a screw-like controller or other control device as is known in the art.

Support tube 125 of access support device 200 is structured to be slidably received through the helical coil. Access support device 200 can further comprise pericardial transection device 101 coupled to a distal end of a catheter 129 structured to be traversed through support tube 125 and the helical coil for introduction into a cavity or space, such as a pericardial cavity, aorta, atrial appendage, or coronary sinus, for example.

In one example, sheath 130 comprises a plurality of tapered segments 116 that coalesce together about a guidewire in the first configuration and are structured to separate and provide additional stabilization and/or support in the second configuration. In one example, the plurality of tapered segments are separated by the support tube 125 in the second configuration.

In some examples, fluoroscopy is used to visualize the position of the access support device 100, 200, support tube 125, and/or the pericardial transection device 101 as it is advanced towards the heart. Visualization may be enhanced by incorporation of a radiopaque marker randomly distributed or presented in a pattern on or in one or more of the components of the access support device. In one example, the cannula is visualized from the left first oblique view at approximately 35-40°. In one example, the user may inject a contrast solution through the catheter 129 or through a lumen 139 of the support tube 125 during deployment.

Part or all of the medical device (e.g., the transcatheter device, the pericardial incision assembly, the pericardial transection device, access support device, the guidewire and controller) may be sterilized for use. Sterilization includes aseptic sterilization. The medical device may be sterilized using various sterilizing techniques, such as E-Beam sterilization, gamma sterilization, ethylene oxide sterilization, autoclave sterilization, chemical sterilization, and/or the like. Additionally, one or more materials used in the medical device may have anti-bacterial characteristics.

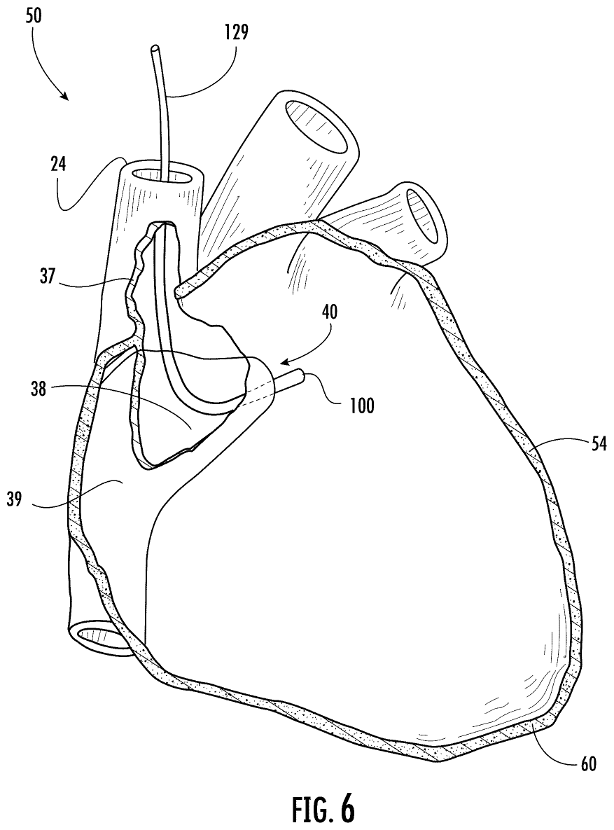

FIG. 6 shows a heart 50 viewed in isolation from the body as an exemplary soft tissue target that is envisaged as using the presently disclosed access support devices presently disclosed. The pericardium 60 or pericardial sac encases the cardiac muscle (i.e., epicardium, myocardium and endocardium). The small space which is present between the heart muscle and pericardium 60 represents the pericardial cavity 54.

The presently disclosed access support devices can be presented to the pericardium 60 in one example via the right atrial appendage 38 (RAA), which is a suitable site for entry into the pericardial cavity 54. Right atrial appendage 38 lies tangential to and between pericardium 60 and the epicardium/epicardial adipose tissue 57. In one example, the presently disclose access support device 100 is guided into right atrial appendage 38 via right atrium 39 so as to be positioned substantially in parallel with the wall of pericardium 60 such that when the wall of right atrial appendage 38 is pierced by the device, it is done substantially without risk of damaging the epicardium or other heart tissue.

Right atrial appendage 38 may be accessed via conventional venae cava routes. FIG. 6 illustrates presentation of access support device 100, 200 into right atrium 39 via the superior vena cava 24. A cut-away 37 shows passage of transection device 101 of access support device 100, 200 through superior vena cava 24, right atrium 39, and right atrial appendage 38. A distal tip of catheter 129 is shown exiting right atrium appendage 38 at apex 40.

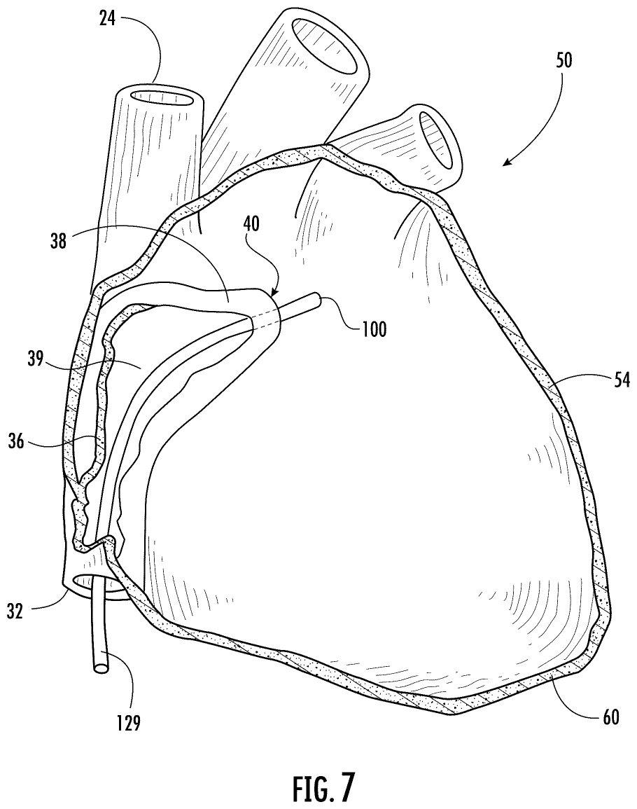

FIG. 7 illustrates an alternative presentation of any of the previously disclosed access support devices into right atrium 39 via the inferior vena cava 32. A cut-away 36 shows passage of catheter 129 through inferior vena cava 32, right atrium 39, and right atrial appendage 38. A distal tip of catheter 129 is shown exiting right atrium appendage 38 at apex 40.

Thus, by way of example, the method of the present disclosure includes the following steps. Access support device 100, 200 is maneuvered downstream through one of the venae cava 24, 32 to right atrium 39. Once presented to the right atrium 39, securement member 146, 246 and/or sealing member 150 secure access support device 100, 200 so as to allow transection device 101 or another device entry into right atrial appendage 38. The wall of right atrial appendage 38 is pierced at apex 40, and the catheter 129 with transection device 101 or other device advanced into the pericardial cavity 54. Other transvascular-right heart routes to the pericardial cavity 54 and other soft tissue targets are envisaged.

Note that the wall of the right atrial appendage may be initially pierced with the access support device 100, 200 itself, or with an instrument (e.g., guidewire) passed through a lumen of the device, e.g., over the wire. Further, any of the previously disclosed devices may be passed into the pericardial space through the opening in the wall of the atrial appendage, or an instrument passed through the lumen of the device may be presented into the pericardial cavity 54. These details will depend on the procedure being performed and on the type of the previously disclosed device being employed.

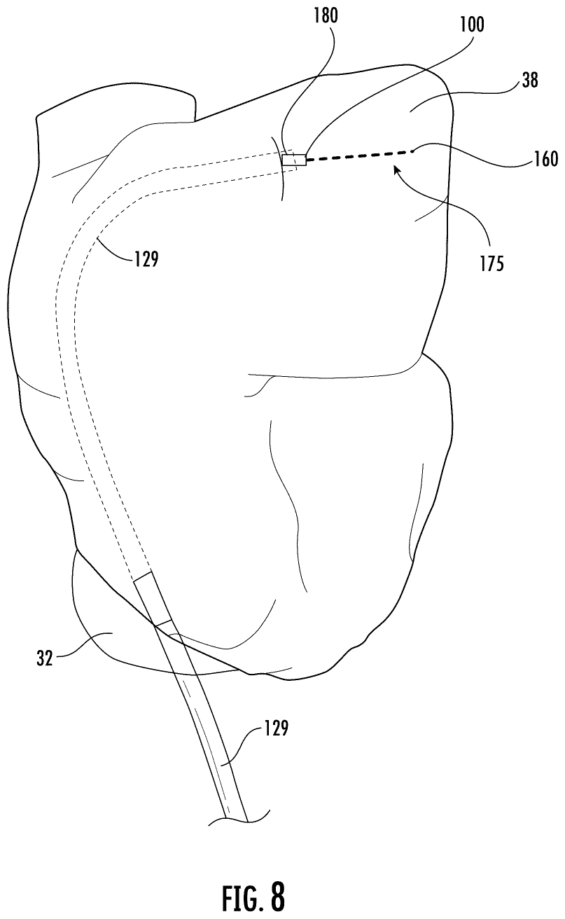

With reference to FIG. 8, use of access support device 100, 200 and subsequent deployment of transection device 101 is shown in a transvascular entry through entry point 180 of the right atrial appendage 38 to the pericardial cavity 54 to a starting point 160 and the creation of incisions 175 in the parietal layer back to the entry point 180. A subxiphoid approach can be used to present access support device 100, 200 to the pericardium 60. Other cut paths and lengths as well as approaches can be used.

Thus, a method of accessing soft tissue, for example, pericardial tissue, for deployment of a pericardial transection device 101 using the presently disclosed access support devices 100, 200 is provided, the method comprising (i) presenting a distal end of access support device 100, 200 to an first surface 55A of a pericardium, (ii) slidably extending the support tube 125 from the access support device 100, 200 and through the portion of the first surface 55A, (iii) presenting the sealing member 150 or the securement member 146, 246 to at least a portion of an opposing surface 55B of the pericardium, and (iv) coupling the access support device 100, 200 to the pericardium. In one example, the method further comprising introducing a pericardial transection device 101 to a pericardial cavity through the support tube 125.

In one example, introducing the pericardial transection device 101 to the pericardial cavity is through the securement member 146, 246. With reference to access support device 100, activating the sealing member 150 involves retracting support tube 125 and compressing a portion of the pericardium between the sealing member 150 and the securement member 146. Support tube 125 and elongated body 130 with transection device 101 can be extended through the securement member 146, for example, through a helical coil for accessing the pericardial cavity 54.

With reference to access support device 200, support tube 125 with securement member 246 is presented to first surface 55A of pericardium and securement member 246 is anchored through the pericardium to the opposing surface 55B. Support tube 125 and elongated body 130 with transection device 101 can be extended through the securement member 146, for example, through a helical coil for accessing the pericardial cavity 54.

In one example, access support device 100, 200 is presented subxiphoidally to the first surface 55A for introducing a pericardial transection device to perform one or more incisions of aligned along a path for providing relief of pericardial restraint. In one example, pericardial access support device 100, 200 is introduced transvascularly to the first surface 55A. By way of example, access support device 100, 200 can be presented transvascularly to the first surface 55A via a Superior Vena Cava, or the access support device 100, 200 can be presented transvascularly to the first surface 55A via an Inferior Vena Cava. In other examples, the access support device 100, 200 can be presented transvascularly to the first surface 55A via a coronary sinus.

In one example, to provide orientational stability of the cutting surface to that of the parietal layer, an OTW introduction is employed for any of the previously disclosed access support devices 100, 200, for example, whether through a dedicated lumen in transcatheter cross-section or ‘Rapid Exchange’ style catheter, or off-center attached cannula, or deflect-resistant catheter, as the delivering catheter.

Access support device 100, 200 can be used in combination with any soft tissue cutting device, such as devices using a blade, current carrying wire, or combination thereof.

Exemplary pericardial transection devices that can be used with the presently disclosed access support device are not limited, and can include, for example, know devices for accessing the pericardium either by the subxiphoid approach or transvascularly.

In one example, the introducer/dilator 115 comprises a puncturing tip as a kit. In one example, at least a portion of the puncturing tip is radiopaque. In one example, at least a portion of the transection device 101 is radiopaque. In one example, at least a portion of the transection device 101 is radiopaque.

In one example, the access support device further comprises at least one nerve proximity device. In one example, the at least one nerve proximity device is a nerve detection device. In one example, the at least one nerve detection device is located on elongated body 130. In one example, the at least one nerve detection device is located adjacent the pericardial transection device 101. In one example, the at least one nerve detection device is located on the introducer/dilator 115. In one example, the at least one nerve detection device provides a proximity indication to at least a portion of a nerve, e.g., the phrenic nerve. The at least one nerve detection device can be an impedance sensor or other electrical field detection device that can provide a visual, audio and/or tactile indication to the user that the pericardial transection device is in proximity to at least a portion of a nerve.

In one example, the at least one nerve proximity device is at least one nerve stimulation device. In one example, the at least one nerve stimulation device is located on the elongated body 130. In one example, the at least one nerve stimulation device is located adjacent the pericardial transection device 101. In one example, the at least one nerve stimulation device is located on introducer/dilator 115. In one example, the at least one nerve stimulation device provides a proximity indication to at least a portion of a nerve, e.g., the phrenic nerve. The at least one nerve stimulation device can be an electrical lead or other electrical discharging device that can provide a visual indication to the user that the pericardial transection device is in proximity to at least a portion of a nerve, e.g., a breathing change of the patient when in proximity to the phrenic nerve.

In one example, the presently disclosed access support devices 100, 200 discussed above further comprise an optical channel in the transcatheter to accommodate a lens coupled to a fiber optic cable, optionally with a light source, e.g., an LED. In one example, the presently disclosed method further comprises obtaining visual information during accessing, traversal of the pericardial cavity 54, exiting and/or cutting, for example, using an optical channel in the transcatheter to accommodate a lens coupled to a fiber optic cable, optionally with a light source, e.g., an LED.

In one example, the presently disclosed access support devices 100, 200 discussed above further comprise signal guided capabilities for presenting to the pericardium. Signal guided capabilities can include echocardiograph, impedance, and electrical signals.

The access support device can be structured such that the total outer diameter (O.D.) is between about 6 Fr (2 mm) and about 30 Fr (10 mm). The support tube 125 of the presently disclosed access support devices 100, 200 can be structured such that the total outer diameter (O.D.) is between about 6 Fr (2 mm) and about 20 Fr (6.67 mm). The catheter 129 of the presently disclosed access support devices 100, 200 can be structured such that the total outer diameter (O.D.) introduced to the pericardial cavity is between about 6 Fr (2 mm) and about 15 Fr (5 mm). The catheter 129 of the presently disclosed access support devices 100, 200 can be structured such that the total outer diameter (O.D.) introduced to the pericardial cavity is between about 6 Fr (2 mm) and about 12 Fr (4 mm). The catheter 129 of the presently disclosed access support devices 100, 200 can be structured such that the total outer diameter (O.D.) introduced to the pericardial cavity is approximately 10 Fr (3.33 mm).

A kit, comprising the pericardial transection device, a sheath, a guidewire 113, and a puncturing tip is provided.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures but may also encompass combinations of elements of the various illustrated examples and aspects thereof.

We claim:

1. A soft tissue access support device comprising:
an elongated body extending along a longitudinal axis between a distal end and a proximal end and defining a first lumen therein;
a sealing member coupled adjacent to the distal end of the elongated body, said sealing member structured to seal to a first surface of a pericardial tissue;
a support tube extending along a longitudinal axis between a distal end and a proximal end and defining a second lumen therein, wherein the support tube is slidably arranged in the lumen of the elongated body between first and second sliding positions;
a securement member coupled to the support tube and structured to engage at least a second opposing surface of the pericardial tissue; and
a guidewire located in the second lumen of the support tube, said guidewire comprising a puncturing tip on a distal end of the guidewire structured to puncture the pericardial tissue.

2. The soft tissue access support device of claim 1, wherein at least the distal end of the elongated body is steerable.

3. The soft tissue access support device of claim 1, wherein at least the distal end of the support tube is steerable.

4. The soft tissue access support device of claim 1, wherein the securement member comprises an anchor.

5. The soft tissue access support device of claim 4, wherein the one or more anchors comprises an inflatable device.

6. The soft tissue access support device of claim 4, wherein the one or more anchors comprises two or more reversibly collapsible-radially expanding shape-memory legs.

7. The soft tissue access support device of claim 4, wherein the one or more anchors comprises a helical coil terminating in a tip structured to engage the pericardial tissue, and upon rotation, urge the anchor into the pericardial tissue.

8. The soft tissue access support device of claim 7, wherein the helical coil of the securement member is structured to helically engage with both the first and the second surfaces of the pericardial tissue.

9. The soft tissue access support device of claim 7, wherein the support member is slidably received through the helical coil.

10. The soft tissue access support device of claim 1, wherein the distal end of the elongated body comprises a plurality of tapered segments that coalesce and which are structured to expand outwardly away from each other upon contact with the support tube.

11. The soft tissue access support device of claim 1, wherein the sealing member is an thermoplastic or thermoset elastomeric, low compression set material.

12. The soft tissue access support device of claim 1, wherein the sealing member is one of an O-ring or an inflatable O-ring.

13. The soft tissue access support device of claim 1, further comprising a transection device coupled to a distal end of a catheter and structured for insertion into the second lumen of the support tube.

14. The soft tissue access support device of claim 13, wherein the transection device comprises a blade or an electrode or combination of a blade and an electrode.

15. The soft tissue access support device of claim 1 further comprising a dilator structured for insertion into the second lumen of the support tube and comprising a hole therethrough to receive the guidewire.

16. The soft tissue access support device of claim 1, further comprising:
a transection device coupled to a distal end of a catheter and structured for insertion into the second lumen of the support tube; and
a dilator structured for insertion into the second lumen of the support tube, wherein the dilator comprises a body extending between opposing ends and having a body width that is equal to or greater than a width of the transection device.

17. A soft tissue access support device comprising:
an elongated body extending along a longitudinal axis between a distal end and a proximal end and defining a first lumen therein;
a sealing member coupled to the distal end of the elongated body, said sealing member structured to seal to a first surface of a pericardial tissue;
a support tube extending along a longitudinal axis between a distal end and a proximal end and defining a second lumen therein, wherein the support tube is slidably arranged in the lumen of the elongated body between first and second sliding positions;
a securement member coupled to the support tube and structured to engage a second opposing surface of the pericardial tissue;
a guidewire located in the second lumen of the support tube, said guidewire comprising a puncturing tip on a distal end of the guidewire structured to puncture the pericardial tissue;
a transection device coupled to a distal end of a catheter and structured for insertion into the second lumen of the support tube; and
a dilator structured for insertion into the second lumen of the support tube, wherein the dilator comprises a body extending between opposing ends and having a body width that is equal to or greater than a width of the transection device.

18. The soft tissue access support device of claim 17, wherein at least the distal end of the support tube is steerable.

19. The soft tissue access support device of claim 17, wherein the securement member comprises a helical coil terminating in a tip structured to engage the pericardial tissue, and upon rotation, urge the anchor into the pericardial tissue, wherein the helical coil of the securement member is structured to helically engage with both the first and the second surfaces of the pericardial tissue.

20. The soft tissue access support device of claim 19, wherein the support member is slidably received through the helical coil.
